(19) United States
(12) Patent Application Publication
(10) Pub. No.: US 2019/0174855 A1
(43) Pub. Date: Jun. 13, 2019

(54) Hat and Eye Covering Combination Assembly

(71) Applicant: Charles D'ambrosio, Highland, NY (US)

(72) Inventor: Charles D'ambrosio, Highland, NY (US)

(21) Appl. No.: 15/840,891

(22) Filed: Dec. 13, 2017

(51) Int. Cl. A42B 1/06; A61F 9/04

(52) U.S. Cl. CPC A42B 1/065; A61F 9/045

(57) ABSTRACT

A hat and eye covering combination assembly includes a hat including a crown with a top wall and a perimeter wall extending downwardly from the top wall. A brim is attached to the crown. A panel comprising a flexible material is attached to the hat. The panel is positionable in a stored position within an interior space bounded by the crown or in a deployed position extending outwardly of the interior space. The panel is positionable in front of a person's eyes when in the deployed position to shield the person's eyes from light and distractions. The panel comprises an opaque material.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to head covering devices and more particularly pertains to a new head covering device for covering a person's eyes.

### BRIEF SUMMARY OF THE INVENTION

An embodiment of the disclosure meets the needs presented above by generally comprising a hat including a crown with a top wall and a perimeter wall extending downwardly from the top wall. A brim is attached to the crown. A panel comprising a flexible material is attached to the hat. The panel is positionable in a stored position within an interior space bounded by the crown or in a deployed position extending outwardly of the interior space. The panel is positionable in front of a person's eyes when in the deployed position to shield the person's eyes from light and distractions. The panel comprises an opaque material.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a bottom side view of a hat and eye covering combination assembly according to an embodiment of the disclosure.

FIG. 2 is a side view of an embodiment of the disclosure.

FIG. 3 is a front view of an embodiment of the disclosure.

FIG. 4 is a bottom side view of an embodiment of the disclosure.

FIG. 5 is a side view of an embodiment of the disclosure.

### DETAILED DESCRIPTION OF THE INVENTION

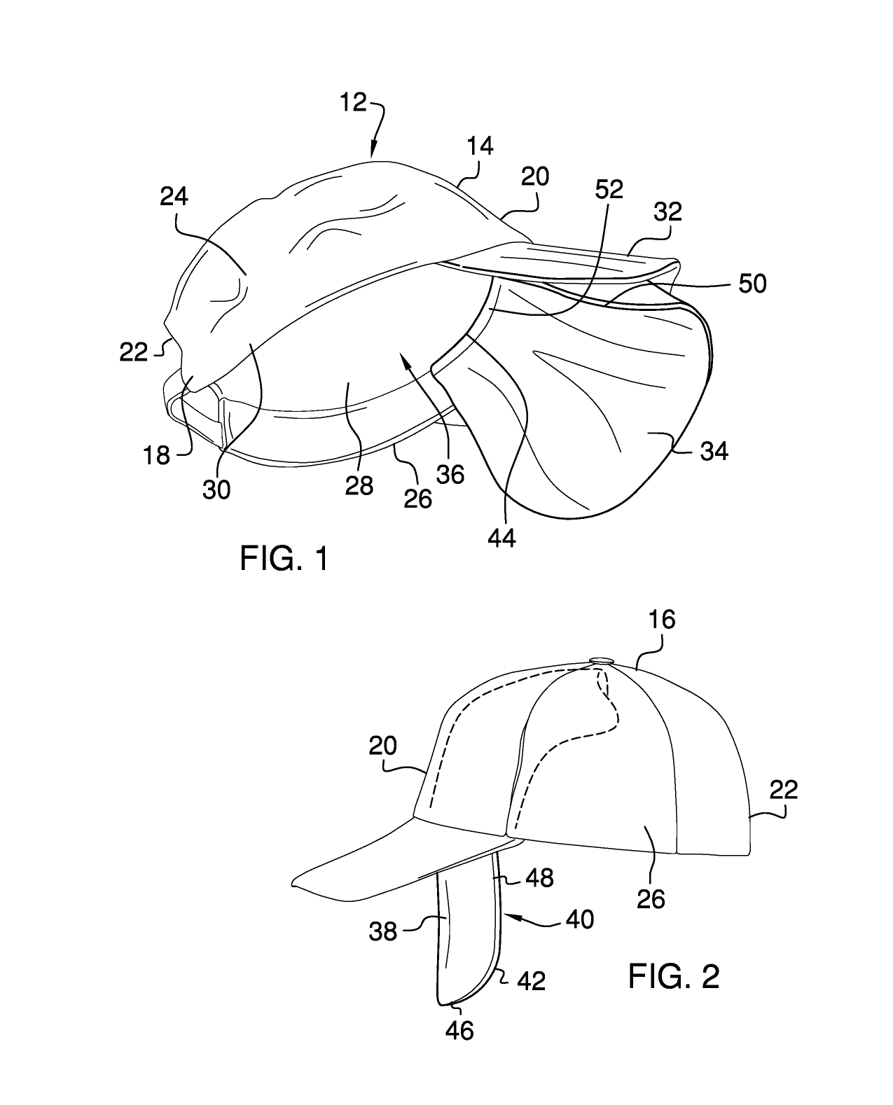

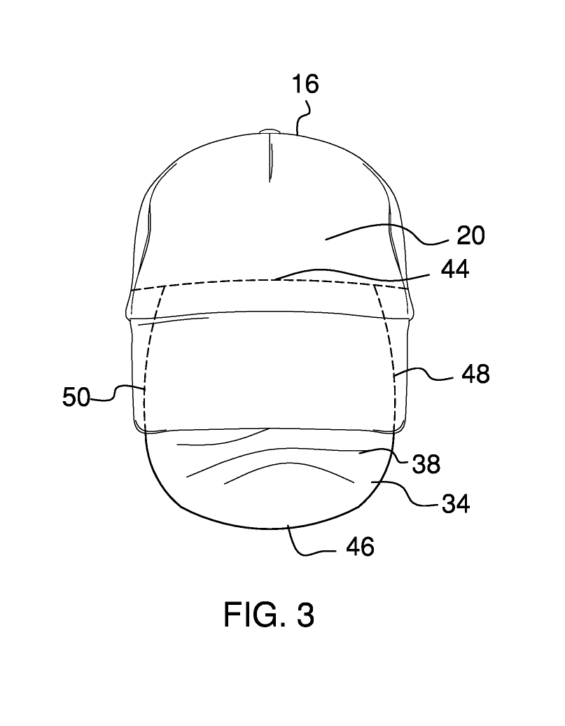

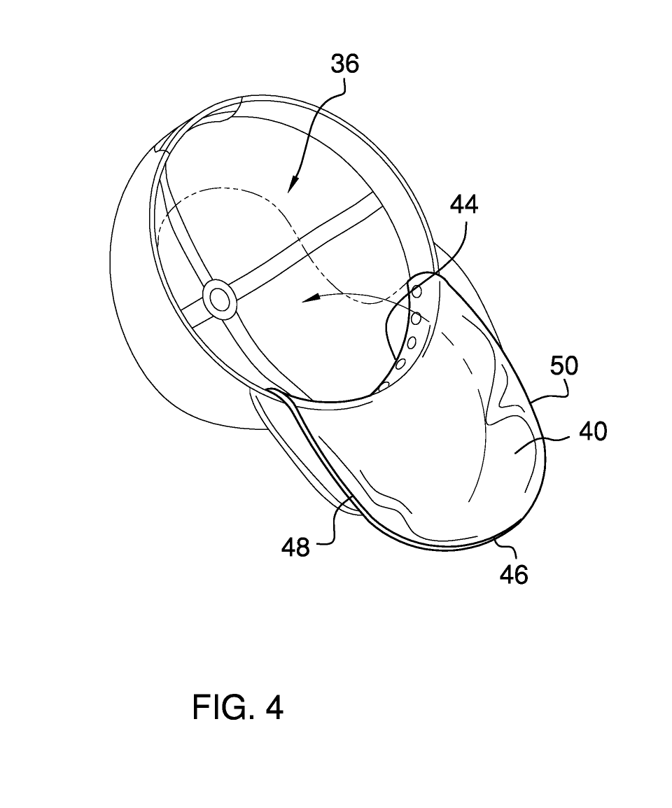

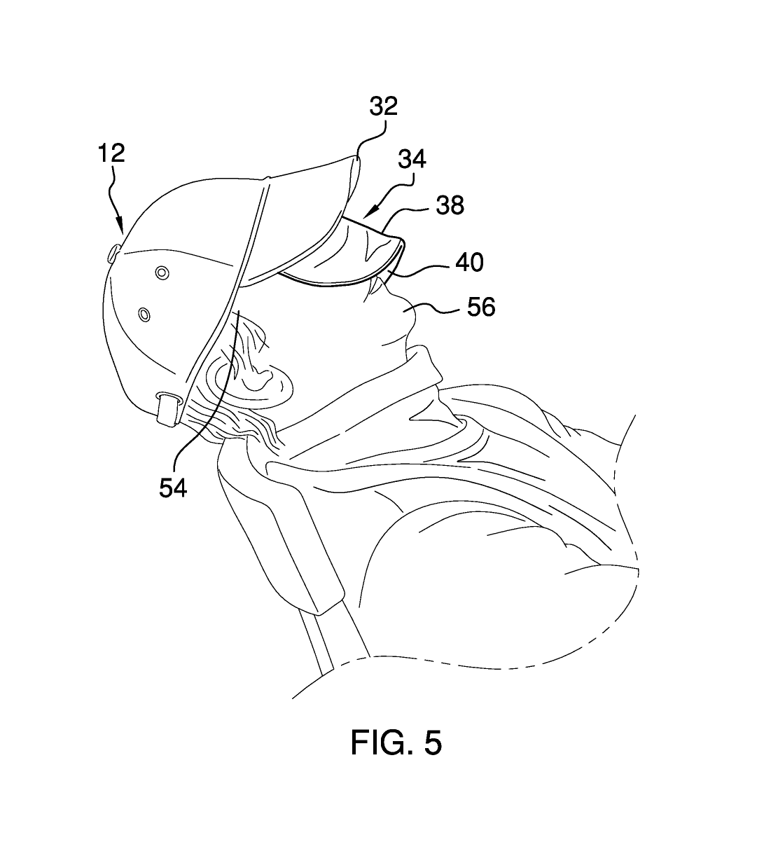

With reference now to the drawings, and in particular to FIGS. 1 through 5 thereof, a new head covering device embodying the principles and concepts of an embodiment of the disclosure and generally designated by the reference numeral 10 will be described.

As best illustrated in FIGS. 1 through 5, the hat and eye covering combination assembly 10 generally comprises a hat 12 that includes a crown 14. The crown 14 has a top wall 16 and a perimeter wall 18 that extends downwardly from the top wall 16. The perimeter wall 18 has a forward side 20, a rear side 22, a first lateral side 24 and a second lateral side 26, an inner surface 28 and an outer surface 30. A brim 32 is attached to the crown 14 and extends outwardly from the outer surface 30 from the forward side 20. The hat 12 may comprise a baseball cap wherein the brim 32 extends exclusively forward of the crown 14.

A panel 34 that comprises a flexible material is attached to the hat 12. The panel 34 is positionable in a stored position within an interior space 36 bounded by the crown 14 or in a deployed position wherein the panel 34 extends outwardly of the interior space 36. The panel 34 is positionable in front of a person's eyes when in the deployed position. The panel 34 has a first surface 38, a second surface 40 and an outward edge 42 that extends therebetween. The outward edge 42 includes a top edge 44, a bottom edge 46, a first lateral edge 48 and a second lateral edge 50. The panel 34 has an upper portion 52 that extends downwardly from the top edge 44. The upper portion 52 may be removably attached to the forward side 20 of the inner surface 28 or non-removably attached to the forward side 20 of the inner surface 28. In the removably attached embodiment the panel may be secured to the cap 12 by a plurality of fasteners such as buttons. The second surface 40 abuts the inner surface 28 of the hat 12 and the first surface 38 abuts a person's head 54 in the stored position. The bottom edge 46 faces downwardly from the hat 12 such that the second surface 40 faces toward a person's face 56 in the deployed position. The panel 34 comprises an opaque cloth material to inhibit light from penetrating the panel 34.

In use, the panel 34 is positioned in the deployed position or the stored position then placed on the person's head 54 to be worn. In the deployed positioned the panel 34 inhibits light from striking the person's eyes. In the stored positioned the panel 34 is positioned within the interior space 36 bounded by the crown 14 and the hat 12 is worn conventionally.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A hat and eye cover combination assembly configured to allow selective positioning over a person's eyes to reduce light contact with the eyes, said assembly comprising:
a hat including a crown, said crown having a top wall and a perimeter wall extending downwardly from said top wall, a brim being attached to said crown; and
a panel comprising a flexible material being attached to said hat, said panel being positionable in a stored position within an interior space bounded by said crown or in a deployed position extending outwardly of said interior space, said panel being configured to be positionable in front of a person's eyes when in said deployed position, said panel comprising an opaque material.

2. The hat and eye cover combination assembly according to claim 1, wherein said perimeter wall has a forward side, a rear side, a first lateral side and a second lateral side, an inner surface, an outer surface, said brim extending outwardly from said outer surface from said forward side, said panel being attached to said hat adjacent to said brim.

3. The hat and eye cover combination assembly according to claim 1, wherein said panel has a first surface and a second surface and an outward edge extending therebetween, said outward edge including a top edge, a bottom edge, a first lateral edge and a second lateral edge, said panel having an upper portion extending downwardly from said top edge, said upper portion being removably attached to said forward side of said inner surface, said second surface abutting said inner surface of said hat and said first surface being configured to abut a person's head in said stored position, said bottom edge facing downwardly from said hat such that said second surface is configured to face toward a person's face in said deployed position.

4. The hat and eye cover combination assembly according to claim 1, wherein said panel is comprised of a cloth material.

5. A hat and eye cover combination assembly configured to allow selective positioning over a person's eyes to reduce light contact with the eyes, said assembly comprising:
a hat including a crown, said crown having a top wall and a perimeter wall extending downwardly from said top wall, said perimeter wall having a forward side, a rear side, a first lateral side and a second lateral side, an inner surface, an outer surface, a brim being attached to said crown, said brim extending outwardly from said outer surface from said forward side, said hat comprising a baseball cap having said brim extending exclusively forward of said crown; and
a panel comprising a flexible material being attached to said hat, said panel being positionable in a stored position within an interior space bounded by said crown or in a deployed position extending outwardly of said interior space, said panel being configured to be positionable in front of a person's eyes when in said deployed position, said panel having a first surface and a second surface and an outward edge extending therebetween, said outward edge including a top edge, a bottom edge, a first lateral edge and a second lateral edge, said panel having an upper portion extending downwardly from said top edge, said upper portion being removably attached to said forward side of said inner surface, said second surface abutting said inner surface of said hat and said first surface being configured to abut a person's head in said stored position, said bottom edge facing downwardly from said hat such that said second surface is configured to face toward a person's face in said deployed position, said panel comprising an opaque cloth material wherein to inhibit light from penetrating said panel.
